SEQUENCE LISTING (9 sequences)

SEQ ID NO: 1 - DNA, 2055 bp, Gossypium hirsutum
  source 1..2055: mol_type = other DNA
aggagcaatcggcaaccaatctgaaaggagatggaggcttatccgaatattttcacccaacttatctcgatccgacgcgaaatccaataccaaaattttcggggcgttagacattgaatctgattgcagcaatgctattcgctggttcaaacaaccgcagagtgctccatcttttatccagagcattattctagcctgcctaagacaatgttctggtattaactttgacttcaagcttattcccagagaagccaattcggtcgccgatcagttggccaaatccggcattggtcaagttgaggatctgcttgtggtgactgctctttgagctgggttccgagatgtggctgtcaattctagctgctttatttgtgctcttgtgcatgctgatcggttgaccactgttggccctgtgtttttccagttctttccttttcttgctctccctttttgttccagcttggtcgcttgcatggggcttttcttctttgttggctgttgtttgcagcatttctgtttatgttgcttgttgtaatactttgaattatattttattatacctttaaaaaaatcgactttgacattatagggatggcatagttacaagaatagtaatataaaaaaacaaaataagacacaatagaggaaaatacgaaggggcaggcaaacgtttgcccaaaaatgaaaaatttttaattcaatccttttataaataaagaaatttgtagagagggtcgactcctcaggagaaatctttaaagtgtcatcttcgggaatgtcgactcggtccctcaaccatggaaattattatccctagagaagggttttgccccccttcacgatgagctgtgtatggttcatagaaggacgtttcgaccgtatgtggacgtacgactacttaccaagaggtgacgtgagcatctcatccttggtgagattcaaacccctaacctatggaatatataggccacctgttggcgtatttggtaccacttgagctagctagctccccaatagctaacctacattccataagtcaagggttttgaatcccaccaagggcgagacactcacgtcgaatcttggtggatggtcgtacgtccacgtacagttaaggcgccccacttcataaaccgtacacaactctccacgaatgggggcaaaattctcccccaatgaacaataccttctattgttgagggaccacattggagtccccaaggacgacacttcaaaggcttatccaataagagctgatccctcatcaacaaagttataaattaatatatagtaaaattacgctttgcccctaaaaatgaaaaaaaaattgttagtaccttaaaaatgaaattataaattaatacatgatatttgtaataaaaaaattaatacatgataaatttagagcctgtttgataacccataatgaaatttactatatgaaattctttccacaaaaaaagtatggaaaattataagtaaatagggacctacttatcacttaatagagaattatattcaattgttattattttattatgtttcatttaagattaaattatcttttatcaaataatttaattataatccgtatttaattttaagtaatatgccaaacaaattttataacttaaatttaacacttaattttttaatatttatttttttcaacacttaaatatatactttgatatcttgaaaattagtccctgctaaattttttctaaattcctcgttagacacagaaaaaaaaaagaacaagatacgaaaacaaaaatacgtaaaattctttgtaaaactttgtacttaaatgttaccaacgatatctaatcatgcttgtgatttaagagaaatgtaaaccagtggtaatgaaccagctatatataattctctcttttttttattttttttataaatgataaataaataaataattttaattttaaaaatcttagtttaaatcagtatctataaacagaatccgtcattcaaatacaaatttcttggtttgtggccaacccaaagccaatggctttgcagcaatgtgatgattag

SEQ ID NO: 2 - DNA, 1591 bp, Gossypium hirsutum
  source 1..1591: mol_type = other DNA
gttgaaatcctccaaattaacacacaagtattttaaagaatgtgactctcttttctaaaaatgagatataaaaattgttacatatatataatttaaaaactataaccctaatatataacttcgacacattaaccttaatttgtaattatcccatcattaatttaattactaggtcactttatttaatatctaaaatccaataaacataatttaatataatttaacattttataataataaataattattgttattagatacacgttacttcctttttcccattcttggaaaatgttaagcatattcaatgcatcacataattaatcattaacacacgcatgctaataatttgtcattaaaccaagcaggattcatgattaaaaccaaacttttgtacggtttgaaaacttgtccatggctaagtatagctttaagtttcatatcagatggctggcttcaacttggtcgatgaagacaaaggaattgcacaggcgagtgagtgacaacttctacatttcgtgctcctaagcatcataacaatagatttcaatggatttgaatatatatattattgtatctctttcttaaaaaaaaaaaagtttattacgacatcgaatttttttatttggttaaaataaaaatcctttcaattggaaaaaatgaaagagaaaaaaaaagaaaaagaaaaaataaaatatatttgtgcatttctaacattttagaaaattattatttgcacatattaataacatatttatttatattaagtgttaatgtgtcacatcacatatatcttatgttgacgtgataatatttaaataaatttaaaatactttaaaaataaattaagtttattttataattatttaaataagcgcatcaaaggttcattctgggcgtgattttttaaataatataaatataaaaatatagaaaaaaattaaaaattaaaattttttttaaagatatttgcgaggcattttgccacatcaacatgatgtccacatggcacaccatgtctcgttgtttggttgctctgtcaaccatgtcatcactaaacaacaaaaatagatggaatttttcaacaaaatacagattttattattattactattttgatataacaataggagttctctttcattttgtggtttgaatttactcttatttgggtcgggataatattcaagtaaaattctccctacaatattgattttaaaattcgagtttaatccaaatacctgaagaagaaatttatttccacttctttccatttgtttgtattatttttcttttaatttgtggaacatgtaatagcatggaggccgaacaatccttgctaaacttatttttttttaataatcagttatcgtttttaagctaaatacgtaatacgaagcttaaatttctacaatacattacatctttcatgattcatactatagacgcaaaatgattgaaacttattctccctcgctactccttctcctgtccatgaacacatatcttgattacggctctcagtttcttatataagggggggggggagcatccatctcaactttcaaacagattcattgagtagattaca

SEQ ID NO: 3 - DNA, 1968 bp, Gossypium hirsutum
  source 1..1968: mol_type = other DNA
gatcctttttgttttgatgttccagggaaggacgaaatcctgagaaatcatgttcaactacacaatagtggggactctgagaacccagatttccaggattttgaaggcttagatgagttaaaagcaaatgattcttatattgatttggtaaccatacactcagctactaataacttctctgattcaaacatgctgggacaaggtggttttggacctgtttacaaggtaatacaaatttctataagataattgagatacccaaatcaacaattctgctatatattgaaagaaaatggttttttctcattagggggtacttcctgatggaaagcaagtggctgtcaaacggctttcaaggttctctgagcaaggtgccttggagttcacaaatgaagtcctactgatattgaaacttcaacataaaaaccttgtcaggcttctgggcttctgtgtggatcagcaagaacagttgcttgtttatgaatttatgcccaacagcagccttgatgttgtcctctttggtattgttcttgaactctttatttgttttgagctctttaagtaactggaaattactcgaagtgtttgcttgatacttgtctcttgcattagatccaaagaaacgtgcacaactcaattggagcaaacgtttaaacattattaatggaattgcaaggggaattctttatctacacgaggattcacgacttaggatcatacatagggacctgaaggctagtaacgtgttgctagattttgacatgaacccgaaaatctcagattttggaatggcaagaatctttgctggagcggacagtgaagctaatacagcacgtatagtcggaacatagtaaggcaccgctcttctagtttcagttcatttcctcaaaatttaactattgttcaaagcacatagtgtgatctgctaaataattctttctcaagcatactaaaatctagttcttctttgcattagtggatatatggctccggagtacgccatggagggattgtattcaatcaagtccgatgttttcagctttggagtactcttgcttgagataattacaggcagaaggaatgcagggttcaatcaaaagaaacatgcccaaacccttgttgcatatgtaactttttaatccacagcttgaattacttgcttttcaatttactagacactgttaatgacattgaatcatgatgcaggcatggcatctatggaaagaagggaatgctctggaattgatggatccattactaactgatggattgatggacgaatttttaagattcattcatatcggactgttgtgtgtccaagaagatgcatttgacaggcctaccatgtcatctgttgtggtaatgttaaaaggtgaaactgtgacactaagccaaccacaacaacctgccttctccatgggaagatttactgagcaccatcaaacattcactagaagttgctccaccaatggtttatcaatttcaagtgttgcacctagatgattgaccccgaaactccagtgatgccagagattcaatcttttctttacctgaaggtttctacatgtgatgataacttcatttcacataaatttagtacatactagtgttatcttctttaagatattttgtatgtcataataagtttgtgaccgtgaatcacaattgtttctaaattccaactaacaaaggctgcccttgcagtagccaaagatactattgggctactaatatccaagtagatagcaaaaaccaagccatcaattatgaaccattcaaattcaatcatcatcaatccttccccttttcagatatttttatacgagatattttcatcaagctttgacacaacattcagatatttttcaactctctccattttagaaaaaagcttaaatacccaaagtttggattcatcactctcaactagatccctttcatctaccacacaaacttagctgaaagatc

SEQ ID NO: 4 - DNA, 34 bp, synthetic construct
  source 1..34: mol_type = other DNA
cacctgcaggagcaatcggcaaccaatctgaaag

SEQ ID NO: 5 - DNA, 31 bp, synthetic construct
  source 1..31: mol_type = other DNA
gagctctaatcatcacattgctgcaaagcca

SEQ ID NO: 6 - DNA, 37 bp, synthetic construct
  source 1..37: mol_type = other DNA
cacctgcaggttgaaatcctccaaattaacacacaag

SEQ ID NO: 7 - DNA, 36 bp, synthetic construct
  source 1..36: mol_type = other DNA
gagctctgtaatctactcaatgaatctgtttgaaag

SEQ ID NO: 8 - RNA, 37 nt, synthetic construct
  source 1..37: mol_type = other RNA
cacctctagagatcctttttgttttgatgtcccaggg

SEQ ID NO: 9 - DNA, 29 bp, synthetic construct
  source 1..29: mol_type = other DNA
ctgcagatctttcagctaagtttgtgtgg

(19) United States
(12) Patent Application Publication
(10) Pub. No.: US 2023/0340518 A1
(43) Pub. Date: Oct. 26, 2023

(54) ABIOTIC STRESS INDUCIBLE PROMOTORS FROM COTTON

(71) Applicant: MAHYCO PRIVATE LIMITED, MUMBAI (IN)

(72) Inventors: Vaishali Khanale, Maharashtra (IN); Anjanabha Bhattacharya, Kolkata (IN); Paramita Ghosh, Pune (IN); Bharat Char, Jalna (IN)

(21) Appl. No.: 18/302,992

(22) Filed: Apr. 19, 2023

(30) Foreign Application Priority Data
Apr. 19, 2022 (IN) 202221023062

(51) Int. Cl. C12N 15/82

(52) U.S. Cl. CPC C12N 15/8273

(57) ABSTRACT

A stress inducible promoter having a nucleic acid sequence as in SEQ ID No. 1 or SEQ ID No.2 or SEQ ID No.3 or complements thereof. The promoter can be an abiotic stress inducible promoter. The promoter can be induced by salt, water, cold or heat stress. The promoter can be derived from Gossypium hirsutum. A genetic construct with the promoter operably linked to a heterologous nucleic acid sequence of interest. A recombinant plant expression vector having the genetic construct. A host cell with the promoter or genetic construct or recombinant plant expression vector. A transgenic plant including the host cell where the transgenic plant is a monocot or a dicot plant. A transgenic seed produced from transgenic plant, where the seed is a monocot or a dicot seed. Methods of expressing a heterologous nucleic acid of interest in a plant.

### SEQUENCE LISTING

Applicant submits herewith a Sequence Listing in XML format in compliance with 37 C.F.R. §§ 1.831-1.835 and respectfully requests entry thereof. The Sequence Listing in XML format includes no new matter.

### FIELD OF THE INVENTION

This invention relates to abiotic stress inducible promoters.

### BACKGROUND OF INVENTION

Plants constantly encounter a wide variety of environmental stresses which can limit their productivity. Stress can be broadly categorized as biotic stress and abiotic stress. Biotic stress can be induced by attack by several pathogens. Abiotic stress can include salinity, drought, flood, extremes in temperature, heavy metals, radiation etc. Abiotic stress is one of the primary reasons for crop loss worldwide. With the onset of global warming, desertification of land, extreme climate change etc., the crop loss due to abiotic stresses is going to increase over time which may severely impact food production worldwide. For the improvement of crop yield and assurance of global food security genetic engineering is one of the promising solutions.

Promoters are important component of expression systems as they control the binding of RNA polymerase, transcription factors, repressor and enhancers. RNA polymerase transcribes DNA into mRNA which is translated into functional protein. Thus, promoters play a crucial role in controlling when and where a gene of interest is expressed in a biological system. Literature studies provide an extensive list of promoters from plant or virus origin having differential expression patterns. Such promoters are useful tools for expressing proteins or peptides in transgenic plants or plant cells or alternatively for silencing genes or gene families. These include constitutive promoters, inducible promoters, developmentally regulated promoters, tissue specific promoters. While generating a transgenic plant, it is desirable to use plant-based promoters as they contain transcription factors binding motifs which induce and drive the expression of gene in a positive manner.

Constitutive promoter such as CaMV 35S promoter is one of the most widely used strong promoter for inducing an expression of foreign gene which have been artificially inserted into a host plant. The “35S promoters” of the cauliflower mosaic virus (CaMV) from isolates CM 1841 (Gardner et al. 1981), Cabb B-S (Franck et al. 1980), Cabb B-JI (Hull and Howell 1978) and the 35S promoter described by Odell et al. 1985 are being used. However, for the development of transgenic crop plants the promoters derived from viruses are less preferred for the transformation of host plant species, as infection of the plants with the virus may cause silencing of the transgene (Seemanpillai et al. 2003, Al-KafFef al. 2000). Also, it is observed that the promoter activity of CaMV35S promoter may get inhibited under stress condition. Michiel Theodoor Jan De Both and coworkers found that the activity of the CaMV 35S promoter in transgenic plants was sensitive to heat stress (WO 2007069894 A2). Stress tolerance is a complex, quantitative and multigeneic trait and to achieve the stress tolerance stacking of tolerant genes would be a potential solution. Therefore, for stacking approach multiple stress inducible promoters are needed, as the use of several identical promoters may result in gene silencing (Yang et al. 2005). Further, stress inducible plant promoters would be advantageous over constitutive promoters while developing stress tolerant crops, as constitutive expression of stress protecting proteins may hinder the growth and development of plants (Kasuga et al. 1999; Li et al. 2013; Rerksiri et al. 2013).

Numerous promoters that function in plant cells and regulates constitutive or stress inducible gene expression in heterologous system are known to those skilled in the art. However naturally occurring promoters increases the basket of promoters available for designing various constitutive or stress inducible promoter options. Further, diverse promoters will ensure the absence of gene silencing due to promoter duplication i.e., multiple copies of the same promoter may cause DNA methylation and thus unexpected pattern of gene expression.

### SUMMARY OF THE INVENTION

The present invention relates to a stress inducible promoter comprising a nucleic acid sequence as set forth in SEQ ID NO. 1 or complements thereof. The promoter can be an abiotic stress inducible promoter. The promoter can be induced by salt, water, cold or heat stress.

The present invention provides a stress inducible promoter comprising a nucleic acid sequence as set forth in SEQ ID NO. 2 or complements thereof. The promoter can be an abiotic stress inducible promoter. The promoter can be induced by salt, water, cold or heat stress.

The present invention provides a stress inducible promoter comprising a nucleic acid sequence as set forth in SEQ ID NO. 3 or complements thereof. The promoter can be an abiotic stress inducible promoter. The promoter can be induced by salt, water, or cold stress.

The present invention provides a promoter having SEQ ID No. 1 or SEQ ID No.2 or SEQ ID No.3 where the promoter can be derived from Gossypium hirsutum.

The present invention provides a genetic construct comprising the promoter having SEQ ID No. 1 or SEQ ID No.2 or SEQ ID No.3 operably linked to a heterologous nucleic acid sequence of interest. The present invention provides a recombinant plant expression vector comprising the said genetic construct. The present invention provides a host cell comprising the promoter having SEQ ID No. 1 or SEQ ID No.2 or SEQ ID No.3 or said genetic construct or said recombinant plant expression vector.

The present invention provides a transgenic plant comprising the said host cell where the transgenic plant is a monocot or a dicot plant. The present invention provides a transgenic seed produced from said transgenic plant, where the transgenic seed is a monocot or a dicot seed.

The present invention provides a method of expressing a heterologous nucleic acid of interest in a plant comprising introducing the genetic construct comprising the promoter having SEQ ID No. 1 or SEQ ID No.2 or SEQ ID No.3, operably linked to a heterologous nucleic acid sequence of interest, where the promoter is induced by an abiotic stress. In the said method, the promoter having the nucleic acid sequence as set forth in SEQ ID NO:1, SEQ ID No. 2 or complements thereof is induced by salt, water, cold or heat stress. In the said method, the promoter having the nucleic acid sequence as set forth in SEQ ID NO.3 or complements thereof is induced by salt, water, or cold stress. In the said method, the heterologous nucleic acid of interest is expressed in the whole plant.

### BRIEF DESCRIPTION OF THE DRAWINGS

FIG. 1 shows pMDCP3 promoter vector (Vector map of pMDC P3 promoter)

FIG. 2 shows Histochemical GUS expression pattern of P3 (SEQ ID NO 1). GUS staining is visible in a whole plant of Arabidopsis.

FIG. 3 shows Histochemical GUS expression pattern of P32 (SEQ ID NO 2). GUS staining is visible in a whole plant of Arabidopsis.

FIG. 4 shows Histochemical GUS expression pattern of P44 (SEQ ID NO 3) GUS staining is visible in a whole plant and parts of Arabidopsis.

FIG. 5 shows Histochemical GUS expression pattern of P3 after 0 day, 8 days and 15 days of water withdrawal.

FIG. 6 shows Histochemical GUS expression pattern of P32 after 0 day, 8 days and 15 days of water withdrawal.

FIG. 7 shows Histochemical GUS expression pattern of P3 after 0 hr, 2 hr and 15 hrs of 150 mM salt stress.

FIG. 8 shows Histochemical GUS expression pattern of P32 after 0 hr, 2 hr and 15 hrs of 150 mM salt stress.

FIG. 9A shows graph of P3, P32 promoter and FIG. 9B shows P44 promoter GUS quantification expression analysis for salt stress assay respectively.

FIG. 10A shows graph of P3, P32 promoter and FIG. 10 B shows P44 promoter GUS quantification expression analysis for water stress assay respectively.

FIG. 11A shows graph of P3, P32 promoter and FIG. 11B shows P44 promoter GUS quantification expression analysis for heat stress assay.

FIG. 12A shows graph of P3, P32 promoter and FIG. 12B shows P44 promoter GUS quantification expression analysis for cold stress assay.

FIGS. 13 to 15 shows PCR results confirming the presence of promoter P3, P32 and P44 in T₁ or T₂ plants.

### BRIEF DESCRIPTION OF THE SEQUENCES

SEQ ID No. 1 labeled as P3 is a promoter sequence of 2055 bp isolated from cotton which is linked with reporter (GUS) drives the expression under different stress conditions.

SEQ ID No.2 labeled as P32 is a promoter sequence of 1591 bp isolated from cotton which is linked with reporter (GUS) drives the expression under different stress conditions.

SEQ ID No.3 labeled as P44 is a promoter sequence of 1968 bp isolated from cotton which is linked with reporter (GUS) drives the expression under different stress conditions.

SEQ ID No. 4 is a PstI forward primer for isolation of P3 promoter.

SEQ ID No.5 is a SacI reverse primer for isolation of P3 promoter.

SEQ ID No.6 is a PstI forward primer for isolation of P32 promoter.

SEQ ID No.7 is a SacI reverse primer for isolation of P32 promoter.

SEQ ID No.8 is a XbaI forward primer for isolation of P44 promoter.

SEQ ID No.9 is a PstI reverse primer for isolation of P44 promoter.

### DETAILED DESCRIPTION OF THE INVENTION

The term “promoter” as used herein is taken in a broad context and refers to regulatory nucleic acid sequences capable of effecting (driving and/or regulating) expression of the sequences to which they are operably linked. A ‘promoter” encompasses transcriptional regulatory sequences derived from a classical genomic gene. Usually, a promoter comprises a TATA box, which is capable of directing the transcription initiation complex to the appropriate transcription initiation start site. However, some promoters do not have a TATA box (TATA-less promoters), but are still fully functional for driving and/or regulating expression. A promoter may additionally comprise a CCAAT box sequence and additional regulatory elements (i.e., upstream activating sequences or cis-elements such as enhancers and silencers). A ‘promoter” may also include the transcriptional regulatory sequences of a classical prokaryotic gene, in which case it may include a −35 box sequence and/or a −10 box transcriptional regulatory sequences. Preferably, the promoter is free of sequences (such as protein encoding sequences or other sequences at the 3′ end) that naturally flank the promoter in the genomic DNA of the organism from which the promoter is derived. Further preferably, the promoter is also free of sequences that naturally flank it at the 5′ end. The promoter may comprise less than about 2 kb, 1.6 kb, 1.2 kb, 1 kb, 0.8 kb, 0.5 kb or 0.1 kb of nucleotide sequences that naturally occur with the promoter in genomic DNA from the organism of which the promoter is derived. The invention encompasses an isolated nucleic acid as mentioned above, capable of regulating transcription of an operably linked nucleic acid in a plant or in one or more particular cells, tissues or organs of a plant.

“Driving expression” as used herein means promoting the transcription of a nucleic acid.

“Regulating expression” as used herein means influencing the level, time or place of transcription of a nucleic acid. The promoters of the present invention may thus be used to increase, decrease or change in time and/or place transcription of a nucleic acid. For example, they may be used to limit the transcription to certain cell types, tissues or organs, or during a certain period of time, or in response to certain environmental conditions.

The term “plant expressible” means being capable of regulating expression in a plant, plant cell, plant tissue and/or plant organ.

A “fragment” as used herein means a portion of a nucleic acid sequence. Suitable fragments useful in the methods of the present invention are functional fragments, which retain at least one of the functional parts of the promoter and hence are still capable of driving and/or regulating expression. Examples of functional fragments of a promoter include the minimal promoter, the upstream regulatory elements, or any combination thereof “Inducible promoters” are responsive to environmental stimuli and provide precise regulation of transgene expression through external control. Inducible promoters are useful for the regulation of potentially stress-related genes that are activated as a result of biotic and abiotic stresses. The differential expression during environmental stimuli helps in meaningful resource utilization.

The term “stress inducible” shall be taken to indicate that expression is predominantly get induced under environmental stress conditions like salt, water, cold and heat. Expression may be driven and/or regulated in the seed, embryo, scutellum, aleurone, endosperm, leaves, flower, calli, meristem, shoot meristem, discriminating centre, shoot, shoot meristem and root.

The term “genetic construct” as used herein means a nucleic acid made by genetic engineering.

The term “operably linked” to a promoter as used herein means that the transcription is driven and/or regulated by that promoter. A person skilled in the art will understand that being operably linked to a promoter preferably means that the promoter is postponed upstream (i.e. at the 5-end) of the operably linked nucleic add. The distance to the operably linked nucleic acid may be variable, as long as the promoter of the present invention is capable of driving and/or regulating the transcription of the operably linked nucleic acid. For example, between the promoter and the operably linked nucleic acid, there might be a cloning site, an adaptor, a transcription or translation enhancer.

The operably linked nucleic acid may be any coding or non-coding nucleic acid. The operably linked nucleic acid may be in the sense or in the anti-sense direction. Typically, in the case of genetic engineering of host cells, the operably linked nucleic acid is to be introduced into the host cell and is intended to change the phenotype of the host cell. Alternatively, the operably linked nucleic acid is an endogenous nucleic acid from the host cell.

The term “transcription terminator” as used in herein refers to a DNA sequence at the end of a transcriptional unit which signals termination of transcription. Terminators are 3′ non-translated DNA sequences usually containing a polyadenylation signal, which facilitates the addition of polyadenylate sequences to the 3′ end of a primary transcript. Terminators active in and/or isolated from viruses, yeasts, molds, bacteria, insects, birds, mammals and plants are known and have been described in literature. Examples of terminators suitable for use in the genetic constructs of the present invention include the Agrobacterium tumefaciens nopaline synthase (NOS) gene terminator (Enrique/et al. 2000), the Agrobacterium tumefaciens octopine synthase (OCS) gene terminator sequence (Jones et al. 1992 and Chung et al. 2005), the Cauliflower mosaic virus (CaMV) 35S gene terminator sequence (Hirt et al. 1990), the Oryza sativa ADP-glucose pyrophosphorylase terminator sequence,t3′Bt2 (Jardinaud et al. 1999 WO1999002687A1), the Zea mays zein gene terminator sequence (Wu et al. 1993), the rbcs-1A gene terminator (Felipe et al. 2020), and the rbcs-3A gene terminator sequences (Kato et al. 2001), amongst others.

An “expression cassette” as meant herein refers to a minimal genetic construct necessary for expression of a nucleic acid. A typical expression cassette comprises a promoter-gene-terminator combination. An expression cassette may additionally comprise cloning sites, for example Gateway recombination sites or restriction enzyme recognition sites, to allow easy cloning of the operably linked nucleic acid or to allow the easy transfer of the expression cassette into a vector. An expression cassette may further comprise 5′ untranslated regions, 3′ untranslated regions, a selectable marker, transcription enhancers or translation enhancers.

The “transformation vector” is a genetic construct, which may be introduced in an organism by transformation and may be stably maintained in said organism. Some vectors may be maintained in for example Escherichia coli, A. tumefaciens, Saccharomyces cerevisiae or Schizosaccharomyces pombe, while others such as phagemids and cosmid vectors, may be maintained in bacteria and/or viruses. Transformation vectors may be multiplied in their host cell and may be isolated again therefrom to be transformed into another host cell. Vector sequences generally comprise a set of unique sites recognized by restriction enzymes, the multiple cloning sites (MCS), wherein one or more non-vector sequence(s) can be inserted. Vector sequences may further comprise an origin of replication which is required for maintenance and/or replication in a specific host cell. Examples of origins of replication include, but are not limited to, the f1-ori and colE 1.

“Expression vector” form a subset of transformation vectors, which, by virtue of having the appropriate regulatory sequences, enable expression of the inserted non-vector sequence(s). Expression vectors have been described which are suitable for expression in bacteria for example E. coli; fungi for example S. cerevisiae, S. pombe, Pichia pastoris or the like; insect cells for example baculoviral expression vectors, animal cells for example COS or CHO cells and plant cells. One suitable expression vector according to the present invention is a plant expression vector, useful for the transformation of plant cells, the stable integration in the plant genome, the maintenance in the plant cell and the expression of the non-vector sequences in the plant cell. The term “selectable marker” includes any gene, which confers a phenotype to a cell in which it is expressed, to facilitate the identification and/or selection of cells that are transfected or transformed. Suitable markers may be selected from markers that confer antibiotic or herbicide resistance. Cells containing the genetic construct will thus survive antibiotics or herbicide concentrations that kill untransformed cells. Examples of selectable marker genes include genes conferring resistance to antibiotics for example nptll encoding neomycin phosphotransferase capable of phosphorylating neomycin and kanamycin, or hpt encoding hygromycin phosphotransferase capable of phosphorylating hygromycin; or herbicides for example bar which provides resistance to Basta; aroA or gox providing resistance against glyphosate; or genes that provide a metabolic trait for example manA that allows plants to use mannose as sole carbon source. Visual marker genes result in the formation of colour for example beta-glucurodinase, (GUS); luminescence for example luciferase or fluorescence for example Green Fluorescent Protein (GFP) and derivatives thereof. Further examples of suitable selectable marker genes include the ampicillin resistance (Ampr), tetracycline resistance gene (tetR), bacterial kanamycin resistance gene (KanR), phosphinothricin resistance gene, and the chloramphenicol acetyl transferase (CAT) gene, amongst others.

The term “transformation” as used herein encompasses the transfer of an exogenous nucleic acid into a host cell, irrespective of the method used for transfer. In particular for plants, tissues capable of clonal propagation, whether by organogenesis or embryogenesis, are suitable to be transformed with a genetic construct of the present invention and a whole plant may be regenerated therefrom. The particular tissue chosen will vary depending on the clonal propagation systems available for, and best suited to, the particular plant species being transformed. Exemplary tissue targets include leaf disks, pollen, embryos, cotyledons, hypocotyls, megagametophytes, callus tissue, existing meristematic tissue (for example apical meristem, axillary buds, or root meristems), and induced meristem tissue (for example cotyledon meristem and hypocotyl meristem). The nucleic acid may be transiently or stably introduced into a plant cell and may be maintained non-integrated, for example, as a plasmid. Alternatively, it may be integrated into the plant genome.

The term “plant” or “plants” as used herein encompasses whole plants, ancestors and progeny of plants and plant parts, including seeds, shoots, stems, roots (including tubers), and plant cells, tissues and organs. The term “plant” therefore also encompasses suspension cultures, embryos, meristematic regions, callus tissue, gametophytes, sporophytes, pollen, and microspores.

The present invention provides abiotic stress inducible plant promoters with differential expression patterns from cotton, thus widening the number and range of promoters that can be used to optimize gene expression and develop products with better precision aimed at abiotic stress tolerance engineering.

The present invention provides stress inducible promoters useful for driving and/or regulating expression of an operably linked nucleic acid in plants. The present invention provides isolating and deriving these promoters from Gossypium hirsutum plant. The present invention therefore concerns promoters, genetic constructs, expression cassettes, transformation vectors, expression vectors, host cells, transgenic plants, transgenic seeds having the promoters according to the present invention. The present invention also concerns methods for expressing a heterologous nucleic acid of interest in a plant.

The present invention provides stress inducible promoters as set forth in SEQ ID No.1, SEQ ID No.2 and/or SEQ ID No.3 isolated from Gossypium hirsutum capable of driving or regulating expression of gene of interest said promoter can be induced by an abiotic stress. The promoters according to the present invention can be induced by salt, water, heat or cold stress. The invention also encompasses fragments of the above-described nucleotide sequences, sequences homologous thereto, altered sequences characterized, by allelic series involving deletions, insertions, or substitutions of one or more nucleotides, either naturally occurring or artificially induced. Suitable variants or complements thereof of SEQ ID No.1 or SEQ ID No.2 or SEQ ID No.3 encompass homologues which have in increasing order of preference at least 90%, 91%, 92%, 93%, 94%, 95%, 96%, 97%, 98% or 99% sequence identity with the nucleic acids as represented in SEQ ID No.1 or SEQ ID No.2 or SEQ ID No.3. The percentage of identity may be calculated using an alignment program known to a person skilled in the art.

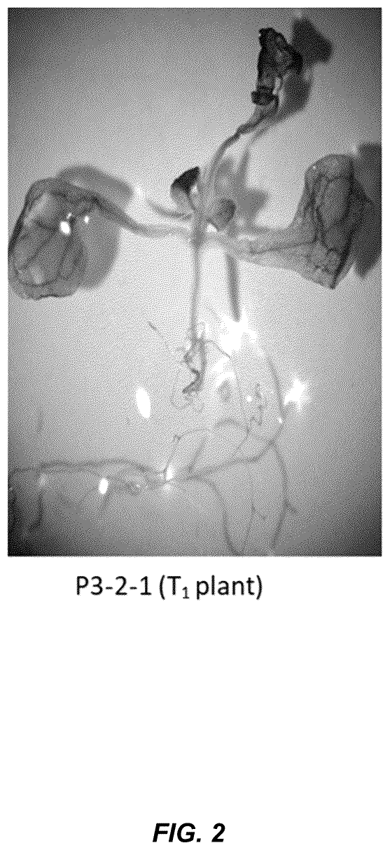

In an embodiment, the present invention relates to a stress inducible promoter comprising a nucleic acid sequence as set forth in SEQ ID NO. 1 or complements thereof. The promoter can be preferably an abiotic stress inducible promoter. The promoter can be induced by abiotic stress such as salt, water, cold and/or heat stress. FIG. 2 shows Histochemical GUS expression pattern of P3 (SEQ ID NO 1). GUS staining is visible in a whole T₁ plant of Arabidopsis, At./pMDC-P3-2-1.

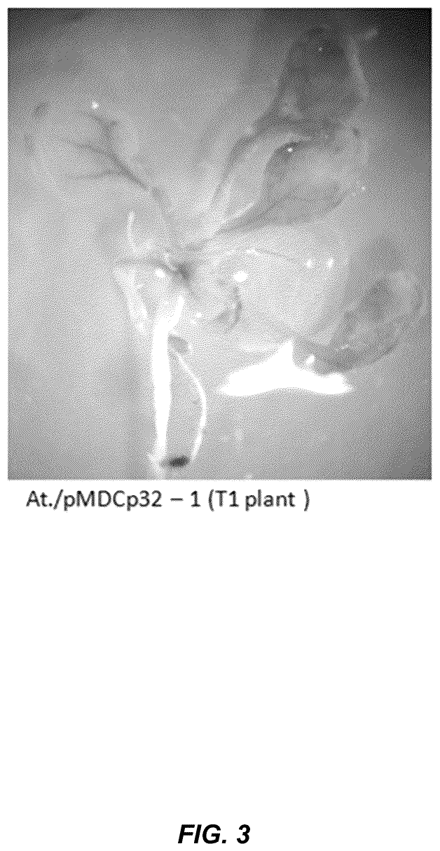

In another embodiment, the present invention relates to a stress inducible promoter comprising a nucleic acid sequence as set forth in SEQ ID No. 2 or complements thereof. The promoter can be preferably an abiotic stress inducible promoter. The promoter can be induced by abiotic stress such as salt, water, cold or heat stress. FIG. 3 shows Histochemical GUS expression pattern of P32 (SEQ ID NO 2). GUS staining is visible in a whole T₁ plant of Arabidopsis, At./pMDC-P32-1.

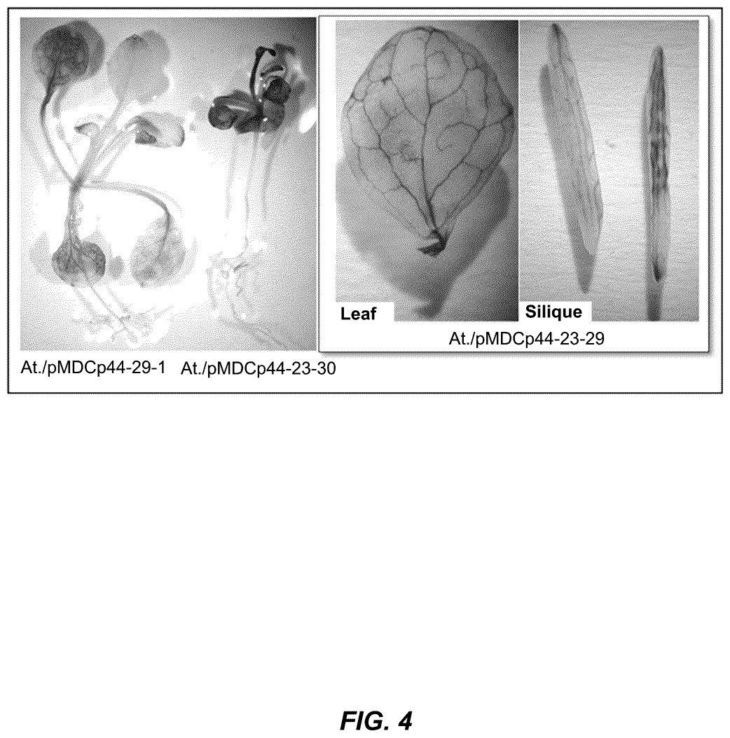

In another embodiment, the present invention relates to a stress inducible promoter comprising a nucleic acid sequence as set forth in SEQ ID No. 3 or complements thereof. The promoter can be preferably an abiotic stress inducible promoter. The promoter can be induced by abiotic stress such as salt, water, or cold stress. FIG. 4 shows Histochemical GUS expression pattern of P44 (SEQ ID NO 3) GUS staining is visible in a whole T₁ plant of Arabidopsis, (At./pMDC-P44-29-1 and At./pMDC-P44-23-30), in leaf and in silique (Magnified view—At./pMDC-P44-23-29).

The promoters of the present invention may comprise cis regulatory elements or motif as depicted in table 7 which can drive the expression of gene under stress conditions.

The promoters as disclosed in any one of SEQ ID No.1 or SEQ ID No.2 or SEQ ID No.3 or the complement thereof can be isolated as nucleic acids of approximately 2 kb upstream of the translation initiation codon (i.e., first ATG) which contains motifs for successfully driving gene expression. Generally, a promoter may comprise from coding sequence to the upstream direction: (i) an 5′ UTR of pre-messenger RNA, (ii) a minimal promoter having the transcription initiation element (Inr) and more upstream a TATA box, and (iii) may contain regulatory elements that determine the specific expression pattern of the promoter. The promoter is preferably a plant-expressible promoter.

In one embodiment, the present invention provides a genetic construct comprising the promoter having SEQ ID No.1 or SEQ ID No.2 or SEQ ID No.3 or complements thereof operably linked to a heterologous nucleic acid sequence of interest.

The genetic constructs of the invention may further comprise a selectable marker and/or an origin of replication. According to some embodiments of the invention, the nucleic acid construct utilized is a shuttle vector, which can propagate both in E. coli (wherein the construct comprises an appropriate selectable marker and origin of replication) and be compatible with propagation in cells. The construct according to the present invention can be, for example, a plasmid, a bacmid, a phagemid, a cosmid, a phage, a virus or an artificial chromosome. In an embodiment, the selectable marker may be GUS: β-glucuronidase (Escherichia coli) GFP: green fluorescent protein (jellyfish, Aequorea victoria), DsRed: Red fluorescent protein (Discosoma spp.) etc. The plant selection marker genes can be hpt (Escherichia coli), nptll (Escherichia coli) bar (bialaphos resistance from Streptomyces hygroscopicus).

The nucleic acid construct of some embodiments of the invention can be utilized to stably or transiently transform plant cells.

Furthermore, the present invention encompasses a host cell having a promoter, or a genetic construct, or an expression cassette, or a transformation vector or an expression vector according to the invention. In particular embodiments of the invention, the host cell can be selected from bacteria, algae, fungi, yeast, plants host cells, preferably plant host cells.

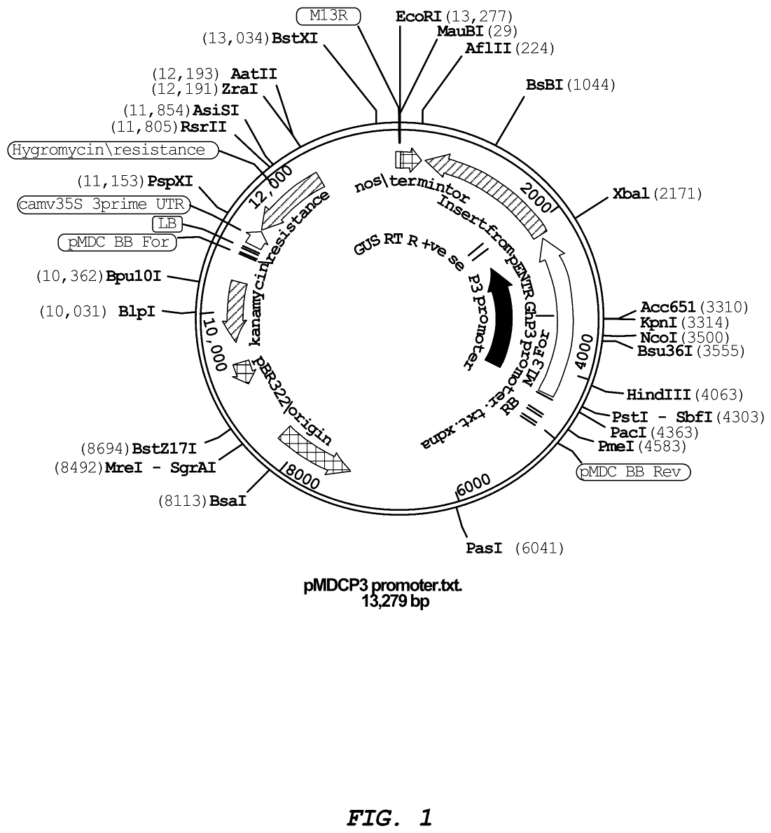

In one embodiment, the present invention provides a plant expression vector. A plant expression vector according to the present invention comprises genetic construct as described here in above. It may further comprise T-DNA regions for stable integration into the plant genome. FIG. 1 shows an example of pMDCP3 promoter vector. Alternatively, plant expression vectors viz., binary vectors, superbinary vectors and gateway vectors can be suitable for Agrobacterium-transformation.

In one embodiment, the present invention provides a transgenic plant cell having a promoter according to the invention, or a nucleic acid, or a genetic construct, or an expression vector according to the invention as described herein above. Preferably said plant cell is a dicot plant cell or a monocot plant cell more preferably a cell of any of the plants as mentioned herein. The dicot plant cell according to the present invention can be Arabidopsis thaliana. Preferably, in the transgenic plant cell according to the invention, the promoter or the genetic construct of the invention is stably integrated into the genome of the plant cell.

In one embodiment, present invention provides a transgenic plant comprising the transgenic plant cell as described herein above. The plant can be a monocot or dicot plant. The transgenic plant may be any monocot or dicot plant of interest, including, but not limited to, plants of commercial or agricultural interest or fundamental research interest such as model plants, crop plants, vegetable plants, fruit plants, flowering plants or ornamental plants. Non-limiting examples of plants of interest include rice, wheat, oat, barley, maize, rye, triticale, tomatoes, eggplants, potatoes, Arabidopsis, sorghum, millet, sugarcane, coconut, oil palm, date palm, olive, tree nuts, canola, cotton, safflower, soybean, sugarbeet, buckwheat, sunflower, tea, coffee, beans, peas, lentils, alfalfa, peanut, lettuce, asparagus, artichoke, celery, carrot, radish, amaranth, cabbages, kales, mustards, and other leafy brassicas, broccoli, cauliflower, Brussels sprouts, turnip, kohlrabi, cucumbers, melons, summer squashes, winter squashes, onions, garlic, peppers, beet, chard, spinach, apple, pear, orange, lime, lemon, grapefruit, apricot, peach, plum, nectarine, banana, pineapple, grape, kiwifruit, papaya, avocado, berries. Preferred monocot plants include, but are not limited to, rice, wheat, oat, barley, maize. Preferred dicot plants include, but are not limited to, cotton, potato, canola, safflower, soybean, sugarbeet, and sunflower, more preferably soybean and cotton. A transgenic seed produced from the transgenic plant of the present invention is also provided. The transgenic seed can be a monocot or dicot seed.

In one embodiment the present invention provides a method of expressing a heterologous nucleic acid of interest in a plant. The method may comprise introducing the genetic construct comprising the promoter having SEQ ID No.1 or SEQ ID No.2 or SEQ ID No.3 or complements thereof, operably linked to a heterologous nucleic acid sequence of interest. The promoter can be induced by an abiotic stress. The heterologous nucleic acid sequence of interest can be any gene that are effective in or needed by a plant during water deficit, cold, heat, salt, or other environmental stress, for plant growth or survival. Potentially any heterologous DNA can be operably linked to the stress-inducible promoter, including genes involved in the environmental stress tolerance, herbicide resistance or tolerance, involved in the enhanced grain quality, enhanced nutrient content, enhanced agronomic traits, male sterility, female sterility, enhanced kernel development, enhanced embryo development and a combination thereof.

The heterologous nucleic acid of interest can be expressed in the whole plant.

Introducing the genetic construct is preferably achieved by transformation. Transformation methods are generally known to a person skilled in the art. They may include the use of liposomes, electroporation, chemicals that increase free DNA uptake, injection of the DNA directly into the plant, particle gun bombardment, and transformation using viruses or pollen and microprojection. Methods may be selected from the calcium/polyethylene glycol method for protoplasts; electroporation of protoplasts; microinjection into plant material; nanoparticles mediated transformation, DNA or RNA-coated particle bombardment infection with (non-integrative) viruses and the like. A preferred transformation method for the production of transgenic plant cells according to the present invention can be an Agrobacterium mediated transformation method.

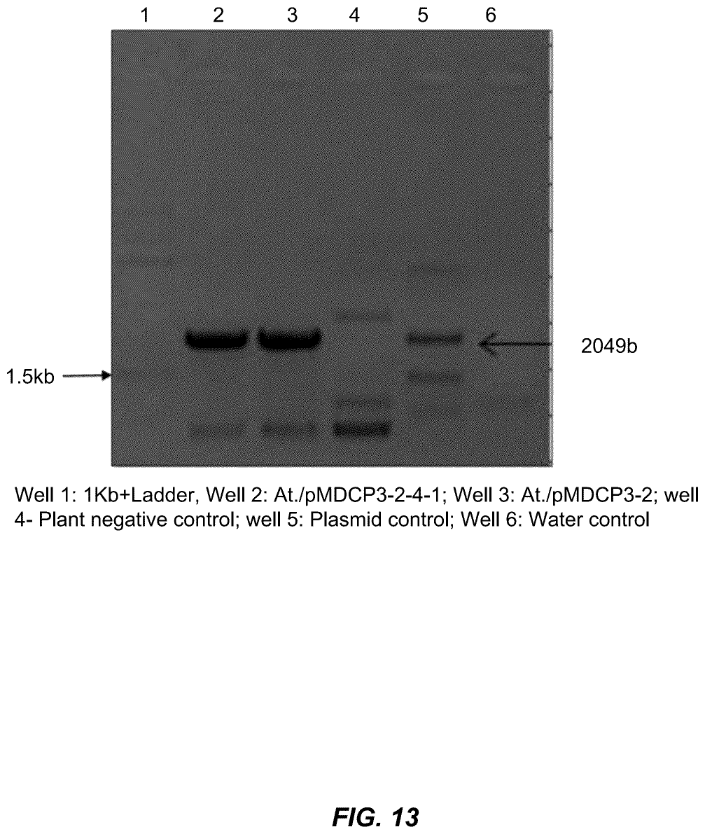

Generally, after transformation, plant cells or cell groupings are selected for the presence of one or more markers which are encoded by plant-expressible genes co-transferred with the gene of interest (which could be under the control of any of the promoters of the present invention). The transformed plants can be cultivated under conditions promoting plant growth. The stability of promoter activity in the next generations or progeny plants of the original plant can be assessed by methods known to person skilled in the art. FIGS. 13 to 15 shows the PCR test results confirming the presence of promoters having SEQ ID No.1 or

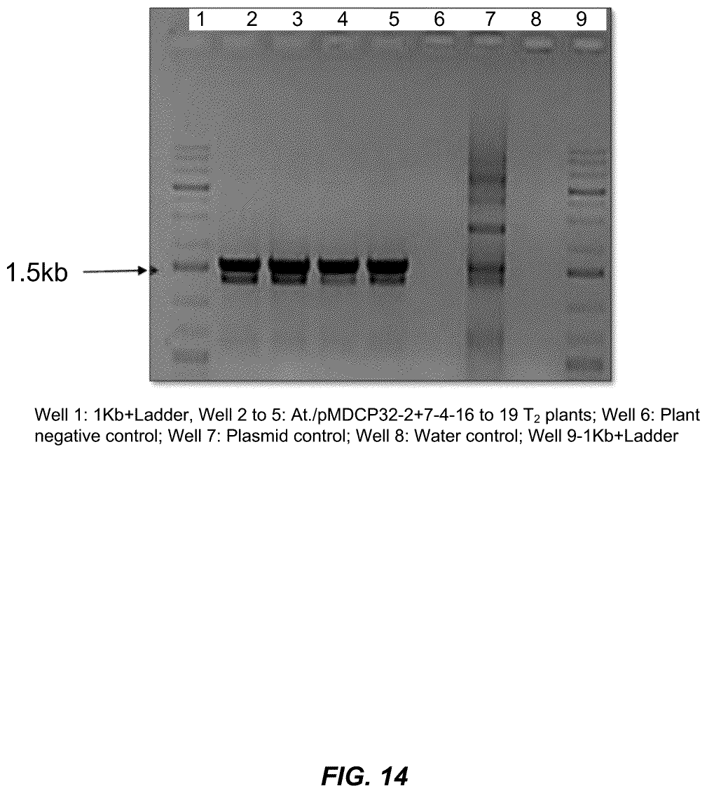

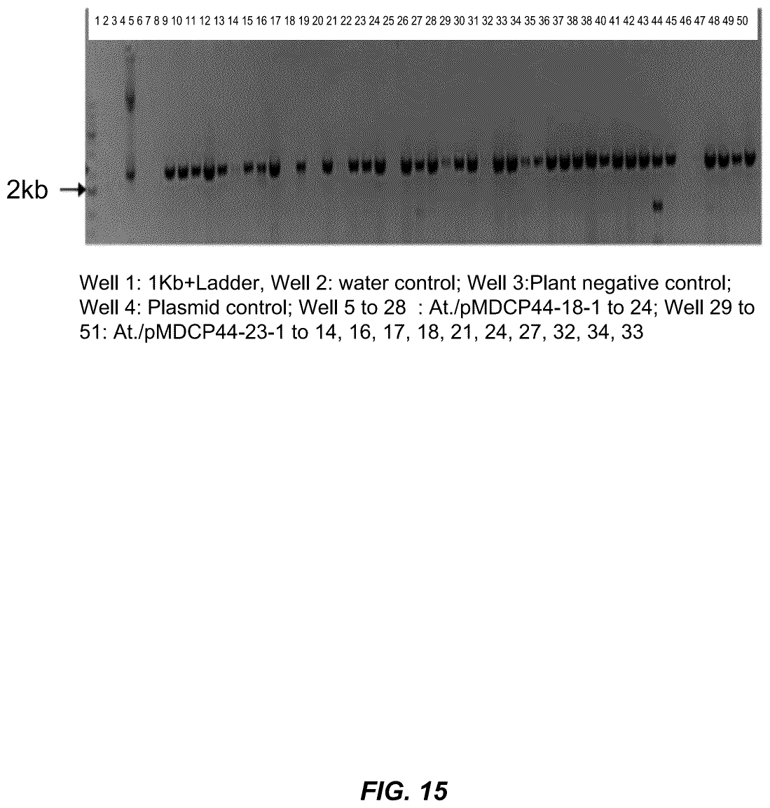

SEQ ID No.2 or SEQ ID No.3 or complements thereof in T₁ or T₂ plants. FIG. 13 shows PCR confirmation of P3 promoter in T₁ plants. FIG. 14 shows PCR confirmation of P32 promoter in T₂ plants. FIG. 15 shows PCR confirmation of P44 promoter in T₁ plants.

The promoter of the present invention can be induced by abiotic stresses. The promoter having SEQ ID No.1 or SEQ ID No. 2 or complements thereof can be expressed under salt, water, cold or heat stress. The promoter having SEQ ID No.3 or complements thereof can be expressed under salt, water, or cold stress. The stress can be induced by various ways.

The effect of the promoters induced by abiotic stress can be determined by the following methods.

Salt Stress:

The plants can be subjected to salt stress by irrigating the plants with a solution containing about 100 to 200 Mm NaCl for about 2 to 15 hours, preferably 2 to 5 hours. Preferably, the plants can be subjected to salt stress by irrigating the plants with a solution containing about 150 mM NaCl for about 2 to 5 hours.

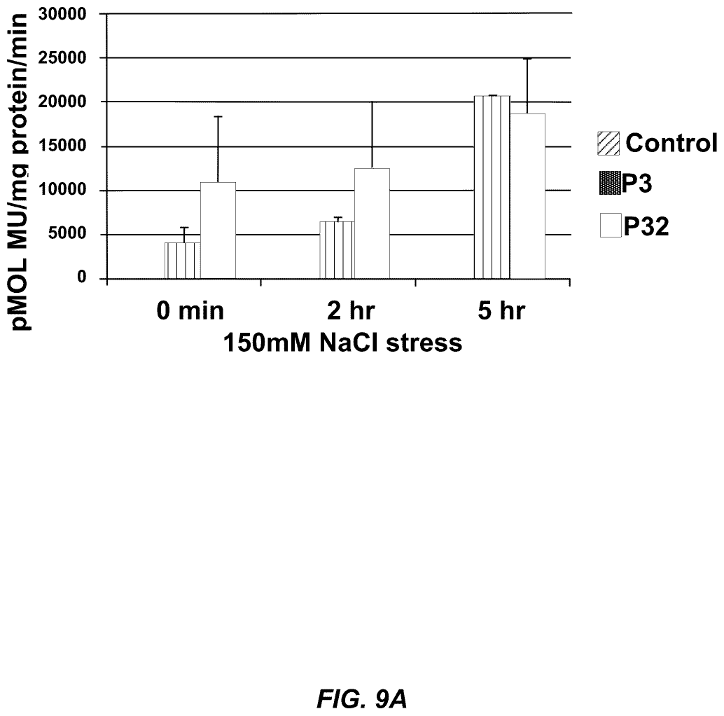

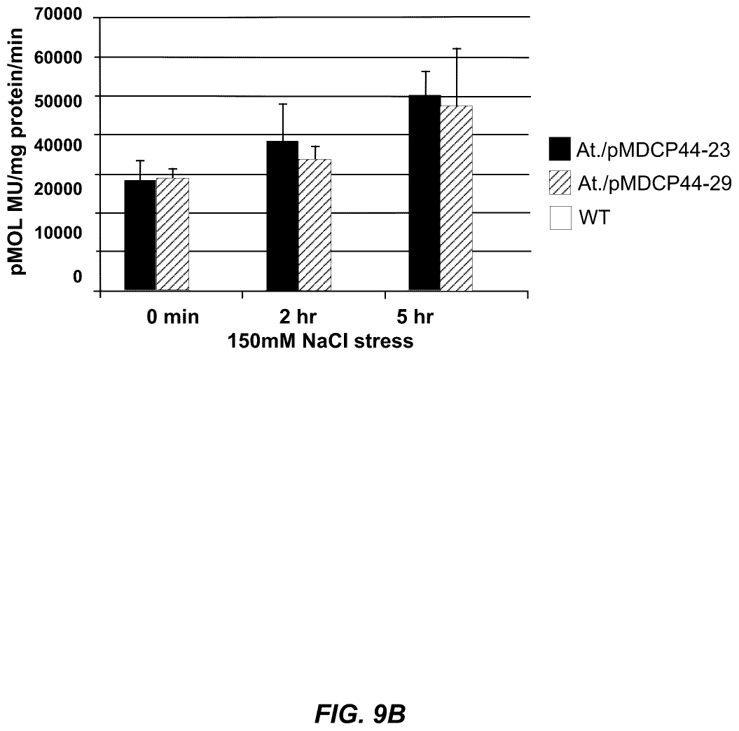

FIGS. 9A and 9B shows graph of P3, P32 promoter and P44 promoter GUS quantification expression analysis for salt stress assay. It can be seen that gradual increase in GUS expression has been observed after exposure of 2 to 5 hours of 150 mM NaCl stress in all three promoters P3, P32 and P44. After 5 hours of 150 mM NaCl stress 4.2 fold, 0.7 fold and 0.8 fold increase in GUS expression has been observed in P3, P32 and P44 promoter respectively.

Water Stress:

Plants can be subjected to water stress by withholding water to the plants for 0-17 days, preferably 8-15 days.

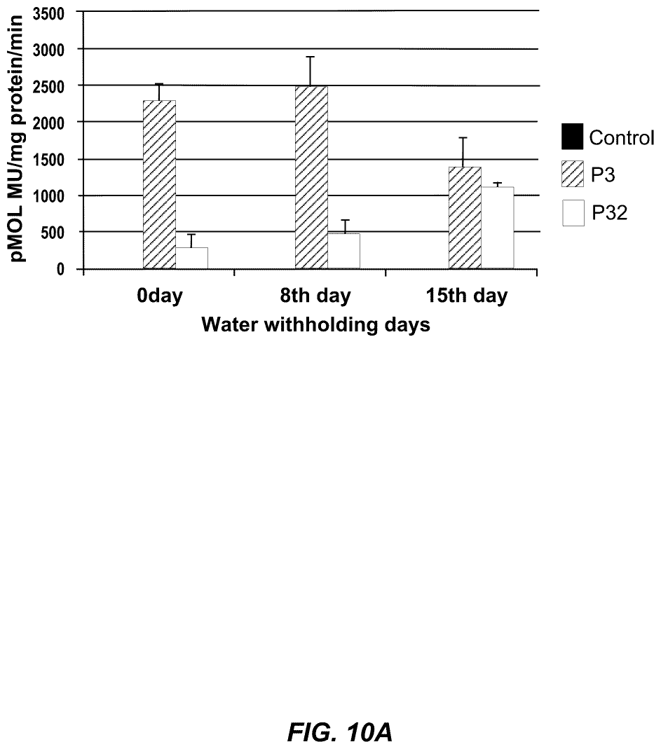

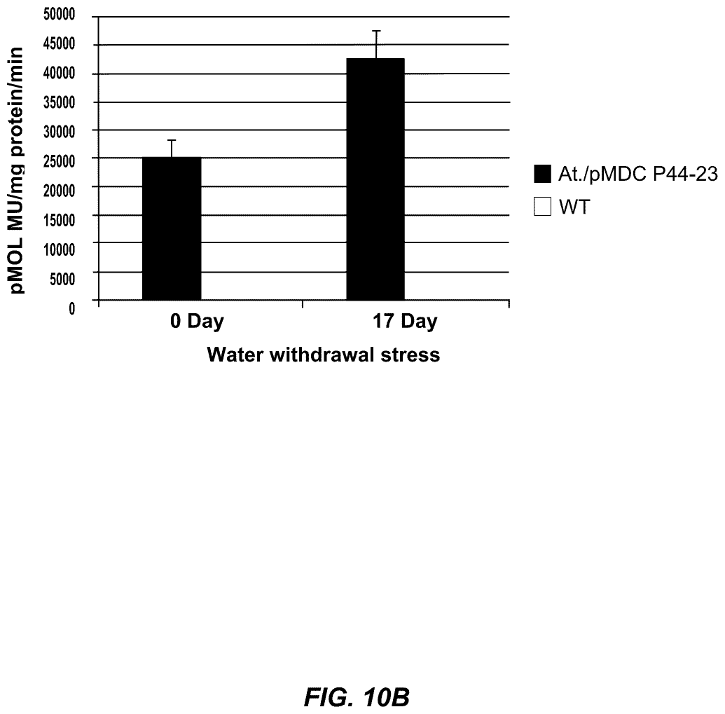

FIGS. 10A and 10B shows graph of P3, P32 promoter and P44 promoter GUS quantification expression analysis for water stress assay. It can be seen that in case of the P3 promoter after 8 days of water stress app. 0.08 Fold increase in GUS expression has been observed and later on after 15 days of water stress GUS expression has dropped down to 0.6 fold and 0.74 fold when compared to 0 day and 8 days of water stress GUS expression respectively.

Gradual increase in GUS expression has observed after 8 days and 15 days of water stress in P32 promoter plants. Around 2.9 fold increase in GUS expression has observed after 15 days of water stress in case of P32 promoter. P44 promoter plants were exposed to water stress for 17 days and around 0.7 fold increase in GUS expression has been observed after 17 days of water stress when compared to day zero expression.

Heat Stress:

The stress can be induced by varying the temperature, for example, the plants can be subjected to heat stress by keeping the plants in an incubator at a temperature of about 35° C. to 42° C. for 2 to 8, preferably 2 to 5 hours each day. Preferably, the plants can be subjected to the heat stress by keeping the plants in the incubator at a temperature of about 35° C. for about 2 to 5 hours.

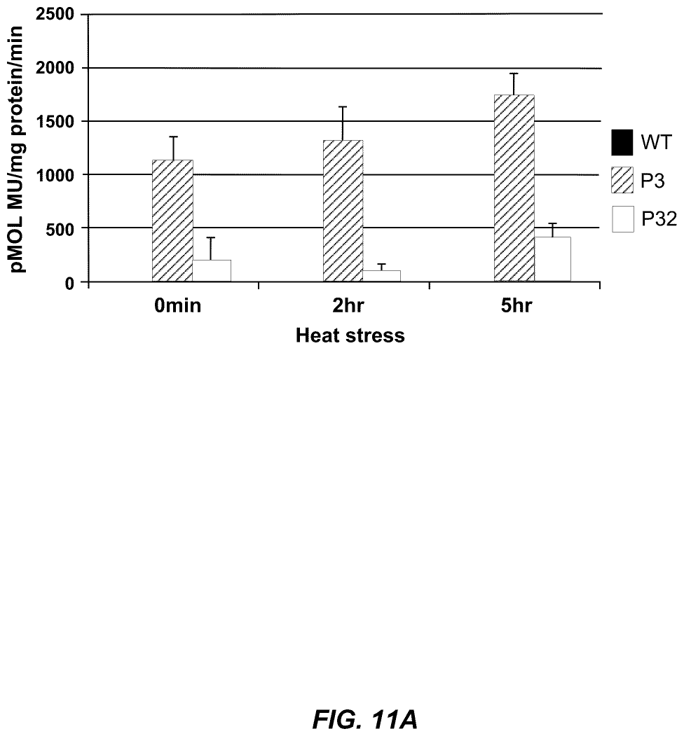

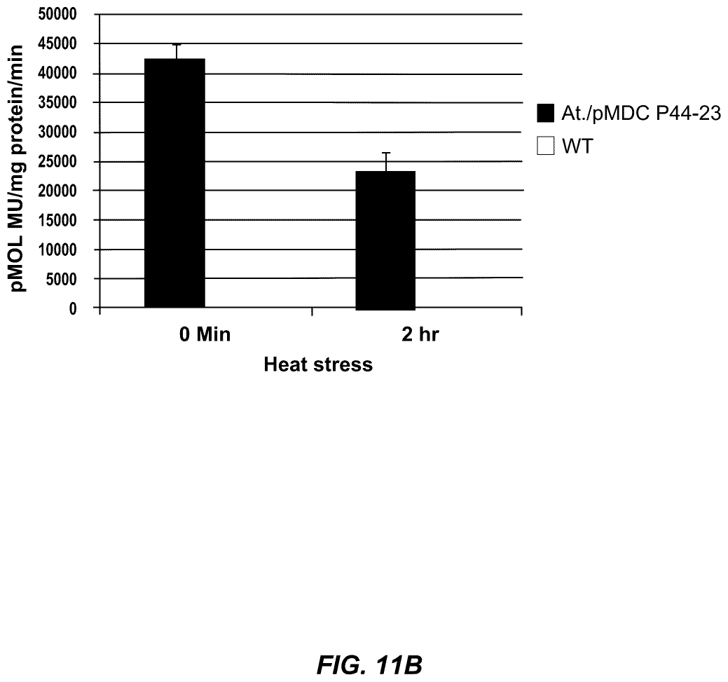

FIGS. 11A and 11B shows graph of P3, P32 promoter and P44 promoter GUS quantification expression analysis for heat stress assay. It can be seen that in case of P3 promoter gradual increase in GUS expression has been observed after 2 and 5 hours of heat stress at 35° C., with 0.54 fold increase after 5 hours of exposure. Around 1.3 fold reduction and 1.04 fold increase in GUS expression was observed in P32 promoter plants when exposed to 2 and 5 hours of 35° C. heat stress respectively, and at 42° C. temperature exposure for 2 hours P44 plants has shown 0.8 fold reduction in GUS expression.

Cold Stress:

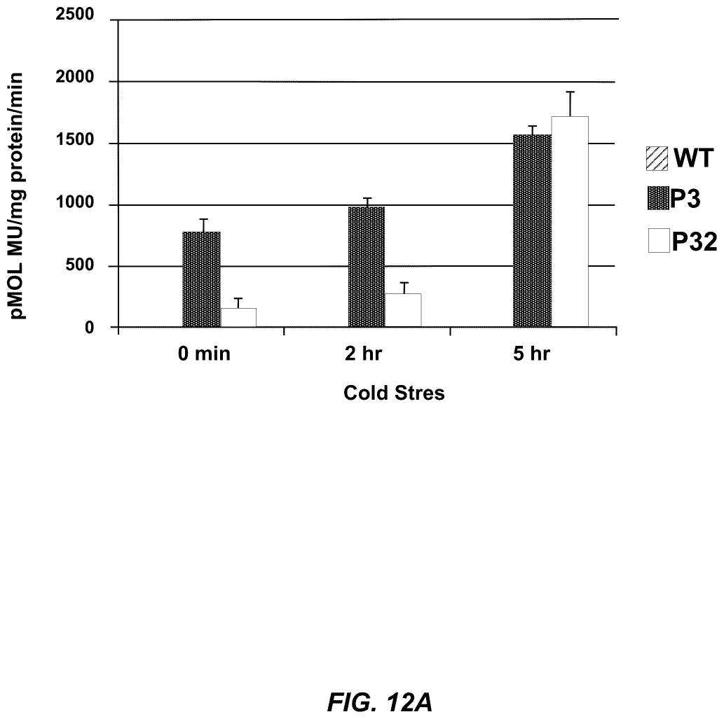

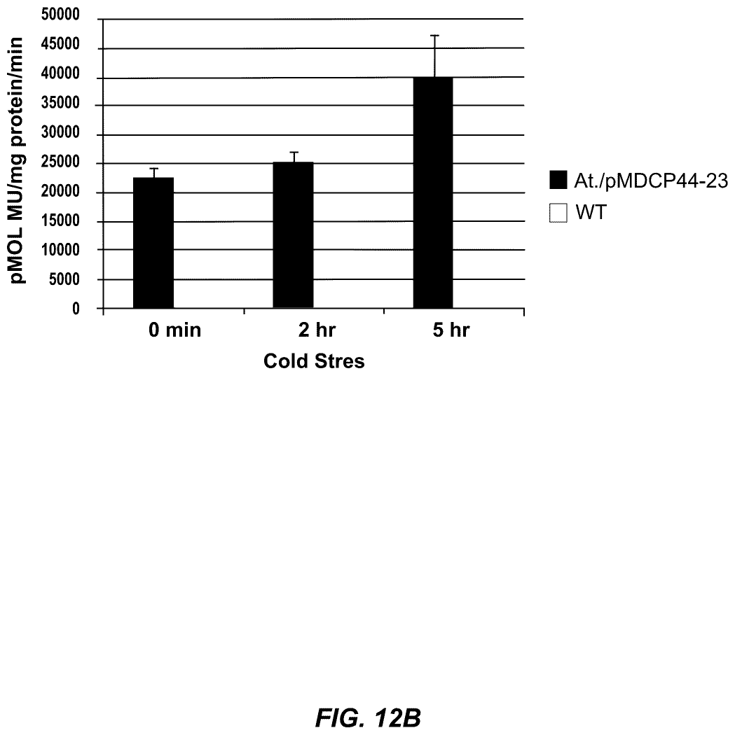

Subjecting the plants to cold stress can be induced by keeping the plants in an incubator at a temperature to 4° C. to 8° C. for 2-8 hours, preferably 2 to 5 hours. Preferably, the plants can be subjected to cold stress by keeping them in an incubator at a temperature of about 4° C. for about 2 to 5 hours each day. FIGS. 12A and 12B shows graph of P3, P32 promoter and P44 promoter GUS quantification expression analysis for cold stress assay. Gradual increase in GUS expression has been observed in all three promoters plants, P3, P32 and P44, when exposed to 2 to 5 hours of 4° C. temperature cold stress. Fold increase of 1.0 fold, 10.27 fold and 0.8 fold increase in GUS expression after 5 hours of cold stress has recorded in P3, P32 and P44 promoters respectively.

The observation of the expression patterns of the isolated nucleic acid sequences, i.e., the promoters of the present invention can be made by visual inspection of the GUS-stained tissues such as roots tissues, leaves, or flower parts (such as anthers). Preferably, sampling can be done once before the start of subjecting the plants to a stress and once after the stress test. A person skilled in the art would understand that the expression of some promoters may be weak in certain tissues and may only be visible with very sensitive detection methods. GUS staining and GUS quantification protocols are known to a person skilled in the art.

The abiotic stress inducible promoters of present invention will be useful in the development of stress tolerant transgenic crops which will be ultimately beneficial to the agriculture eco-system and stakeholders primarily agriculture industries, farmers, and consumers alike.

### EXAMPLES

### Example 1: Identification of Up-Regulated Genes Under Various Stress Condition for Promoter Isolation

An unified gene expression resource like PLEXdb (Plant Expression Database) and GEO (Gene Expression Omnibus) were used to identify highly up regulated probe sets/genes by comparing different abiotic treatments like drought, heat, salinity, and cold microarray experiments. Thus, by carefully considering each and every combination of the selected experiment data, highly expressed eight fold probe set data was generated and redundant probsets were deleted. The probesets having frequency of occurrence three to four times repeatedly were considered for this study. Predicted mRNA sequence and putative promoter sequences were retrieved from CottonGen database (https://www.cottongen.org/). The gene names and putative function of the genes are listed in Table 1.

TABLE 1
Promoter name, PLEXDb probeset ID, CottonGen gene ID, predicted gene and
function

| Sr. | Promoter | PLEXdb | CottoGen | Gene | |
|---|---|---|---|---|---|
| No | ID | ID | ID | name | Published function of the Gene |
| 1 | P3 | Ghi.1624. | Gorai.011G | Hypothetical | Hypothetical protein, |
| | | 1. [symbol] 1_at | 201600.2 | protein | uncharacterized protein of G. |
| | | gb\| | (Uncharacterized | | raimondii, |
| | | DN760793 | protein of | | |
| | | | Gossypium | | |
| | | | raimondii) | | |
| 2 | P32 | Gra.2065. | Gorai.011G | Putative | In plants Cytochrome P450 |
| | | 1.S1_at | 262400.1 | CYTO- | family proteins are involved in |
| | | gb\| | (Uncharacterized | CHROME | the biosynthesis of several |
| | | CO116760 | protein of | P450 | compounds such as hormones, |
| | | | Gossypium | family | defensive compounds, and fatty |
| | | | raimondii) | protein | acids. |
| 3 | P44 | Ghi.10424. | CA993457/ | Predicted | Involved in biosynthesis of the |
| | | 1.S1_s_at | Gorai.010G | THI1 | thiamine precursor-thiazole, |
| | | | 176000.1 | (thiamine | functions in stress adaptation. |
| | | | | thiazole | |
| | | | | synthase) | |

### Example 2: Identification and Isolation of the Promoter Regions of Cotton Genes

The promoter regions of these genes were isolated as the DNA region spanning around 2 kb upstream of the translation initiation codon (i.e., first ATG) which necessarily contains motifs for successfully driving gene expression. The promoter regions were isolated from genomic DNA of Gossypium hirsutum (Coker310 FR line) via PCR using specific primers and high-fidelity DNA polymerases.

PCR conditions for the amplification of promoters are mentioned in Table 2. Primers comprise CACC site for site directed ligation in entry vector and are listed in Table 3. The corresponding PCR fragment was purified from the PCR reaction mix via gel electrophoresis and subsequent purification by HiYield Gel/PCR DNA Mini kit (Real Genomics).

TABLE 2
Promoter amplification PCR conditions

| | | | | | Isolated from |
|---|---|---|---|---|---|
| Sr. | | | | | Gossypium |
| No. | Promoter ID | PCR condition | | | species |
| 1 | P3 | 95° C.-5′ | | | Gossypium |
| | | 94° C.-30″ | | | hirsutum |
| | | 61° C.-30″ | {close oversize brace} | 40X | (cv. Coker |
| | | 72° C.-2′ | | | 310 FR) |
| | | 72° C.-10′ | | | |
| | | 4° C.-∞ | | | |
| 2 | P32 | 95° C.-5′ | | | Gossypium |
| | | 94° C.-30″ | | | hirsutum |
| | | 48° C.-30″ | {close oversize brace} | 35X | (cv. Coker |
| | | 72° C.-2′ | | | 310 FR) |
| | | 72° C.-10′ | | | |
| | | 4° C.-∞ | | | |
| 3 | P44 | 95° C.-5′ | | | Gossypium |
| | | 94° C.-30″ | | | hirsutum |
| | | 49° C.-30″ | {close oversize brace} | 40X | (cv. Coker |
| | | 72° C.-2′ | | | 310 FR) |

72° C.-10′
4° C.-∞

TABLE 3
Primers used for the isolation of promoters

| Sr. | Promoter | SEQ ID | Oligo | | |
|---|---|---|---|---|---|
| No | ID | No. | Name | 5′←SEQUENCE→3′ | Length |
| 1 | P3 | SEQ ID | P3_pstI | CACCTGCAGGAGCA | 34 |
| | | No. 4 | forward | ATCGGCAACCAATC | |
| | | | | TGAAAG | |
| | | SEQ ID | P3_SacI | GAGCTCTAATCATC | 31 |
| | | No. 5 | Reverse | ACATTGCTGCAAAG | |
| | | | | CCA | |
| 2 | P32 | SEQ ID | P32_PstI | CACCTGCAGGTTGA | 37 |
| | | No. 6 | Forward | AATCCTCCAAATTA | |
| | | | | ACACACAAG | |
| | | SEQ ID | P32_SacI | GAGCTCTGTAATCT | 36 |
| | | No. 7 | Reverse | ACTCAATGAATCTG | |
| | | | | TTTGAAAG | |
| 3 | P44 | SEQ ID | P44_XbaI | CACCTCTAGAGATC | 37 |
| | | No. 8 | forward | CTTTTTGTTTTGAT | |
| | | | | GTCCCAGGG | |
| | | SEQ ID | P44_pstI | CTGCAGATCTTTCA | 29 |
| | | No. 9 | reverse | GCTAAGTTTGTGTG | |
| | | | | G | |

### Example 3: Cloning of Promoter-Gus Reporter Vectors for Plant Transformation

The purified PCR fragments corresponding to the promoter regions of the present invention were cloned into the pENTRT/D-TOPO entry plasmid of the Gateway system (Life Technologies) using site specific ligation. The identity and base pair composition of the cloned insert was confirmed by sequencing and additionally, the resulting plasmid was tested via restriction digestion. In order to clone each of the promoter of the present invention in with a reporter gene, “LR recombination reaction” (Gateway) with the destination vector pMDC 164 was performed. This later destination vector was designed to operably link each promoter in the present invention to the Escherichia coli beta-glucuronidase (GUS) gene via the substitution of the Gateway recombination cassette/ccdB gene in front of the gusA gene. Furthermore, this destination vector is suitable for transformation of plants and comprises within the T-DNA left and right borders. The resulting promoter-GUS cassette and selectable marker (Refer Vector map) were thus used for the validation experiments. The resulting reporter vectors, comprising a promoter of the present invention operably linked to GUS, were mobilized into Agrobacterium-strain EHA 105 and subsequently into Arabidopsis plants using transformation techniques as mentioned below.

### Example 4: Arabidopsis Transformation

Agrobacterium—mediated transformation of Arabidopsis thaliana (Columbia-0) was performed using floral dip method as described in Das et al., 2011 with few modifications (Das et al., 2011, Advances in Bioscience and Biotechnology, 2, 59-67).

4.1 Growing of Arabidopsis plants: Arabidopsis—seeds were kept for 3 days at 4° C. to break dormancy. Vernalized seeds were layered on soilrite, in plastic cups. Cups were watered periodically and incubated in growth chamber or in culture room at 25° C. for 16 hr light/8 hr dark condition, up to inflorescence or floral stage. Primary inflorescence was nipped to obtain secondary buds.

4.2 Agrobacterium culture preparation: Two days before the initiation of culture preparation for the floral dip, Agrobacterium strain EHA 105 carrying pMDC164 P3, P32, and P44-GUS was streaked on LB agar with antibiotic selection and incubated at 28° C. (chloramphenicol 10 mg/L and kanamycin 50 mg/L). Before the floral dip experiments, 5 Agrobacterium—culture were scrapped from the plate and was suspended in inoculum 3 media and finally ˜1.0 OD at 600 nm (stationary phase) was used for infection.

4.3 Floral dip: Plants were selected which were in the suitable stage of budding and were dipped in the Agrobacterium culture by agitating for 40 to 50 seconds both vertically and horizontally intermittent shaking. After floral dip plants were kept horizontally in tray cover with polythene bag/Saran wraps for overnight at 25° C. in dark. Next day infected plants were kept upright and incubated at 25° C. for 16 hr light/8 hr dark in culture room till seed harvest.

4.4 Screening of positive transformants: Arabidopsis T₀ seeds were kept for 3 days at 4° C. to break dormancy. Vernalized seeds were sterilized by 1.5% sodium hypochlorite for 1 minute then washed by D/W for 5 times and layered on 0.5×MS without sucrose and with 10 mg/L Hygromycin, incubated at 25° C. for 16 hr light/8 hr dark in culture room.

Media composition used is provided below:
1] Inoculum 3—0.5×MS salt, 1×B5 vitamins, 50 gm/L Glucose, 0.004M BAP, 0.075% Tween-20, pH 5.7
2] LB medium—10 gm/L tryptone, 5 gm/L yeast extract, 10 gm/L NaCl, 8 gm/L Agar, pH 7.0.
3] 0.5× MS—0.5×MS salts and vitamins, 8 gm/L Agar-Agar, pH 5.8

### Example 5: Expression Patterns of the Promoter-GUS Reporter Cassette in Plants: Growth and Harvest of Transgenic Plants or Plant Parts at Various Stages

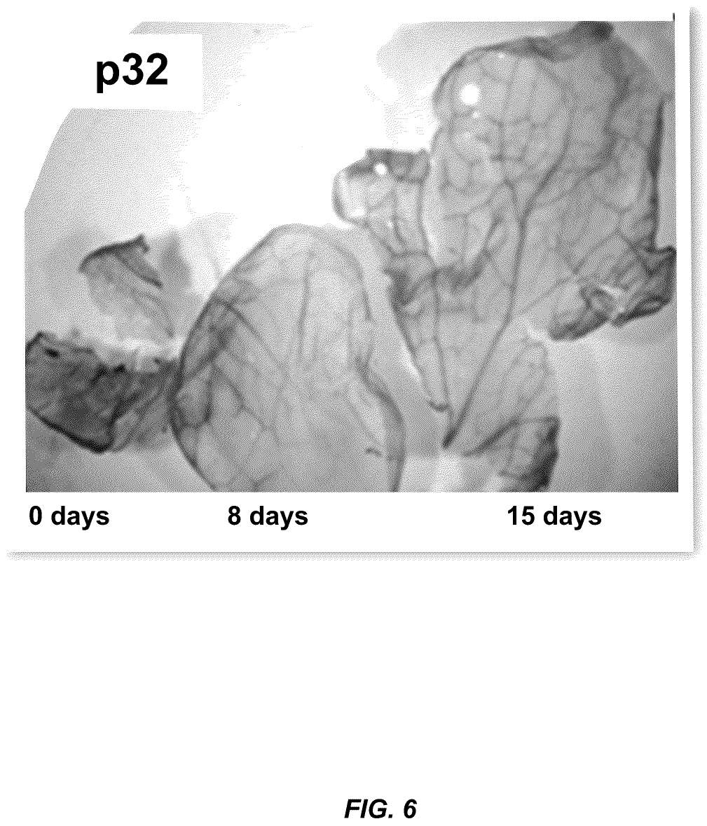

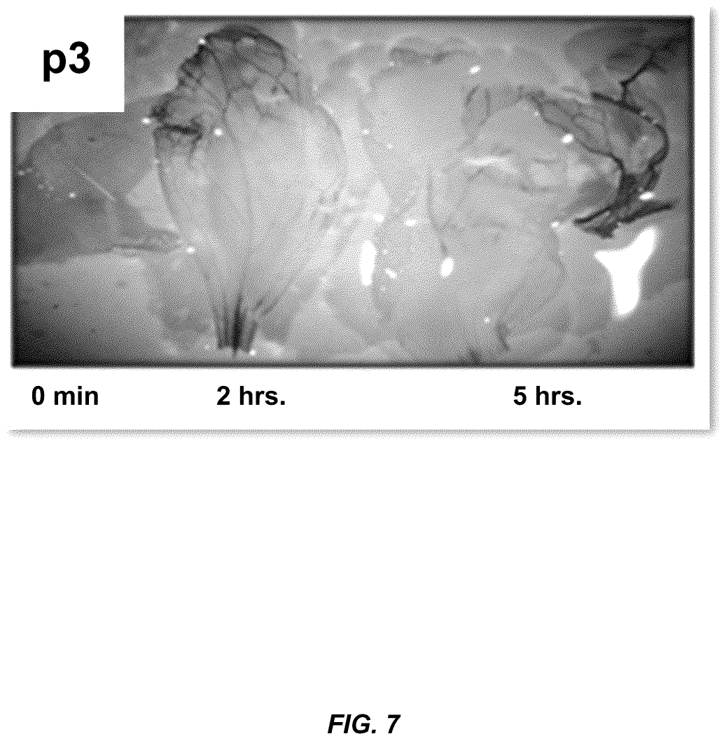

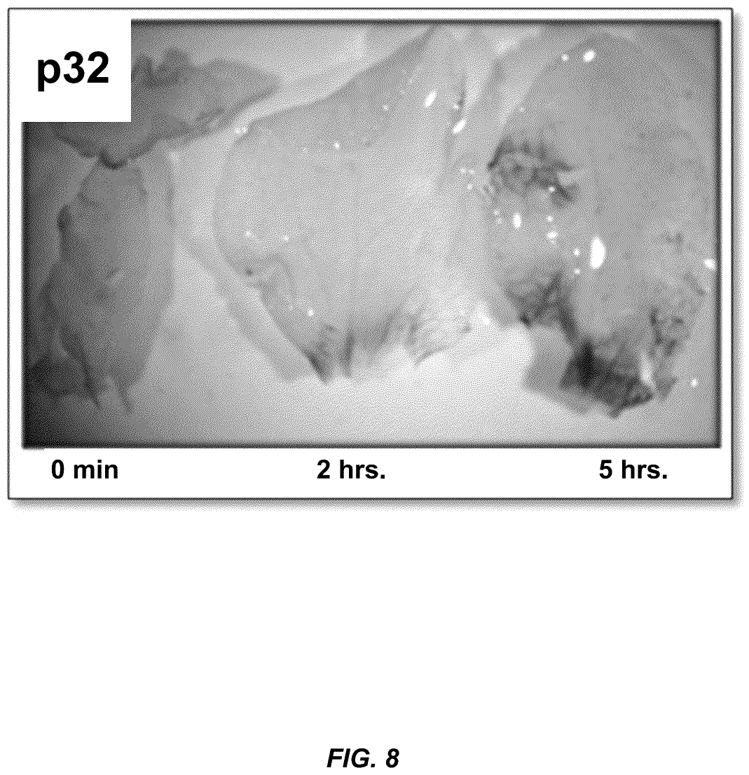

For each promoter-GUS reporter construct T₀ Arabidopsis plants were generated from transformed cells. Plant growth was performed under normal conditions. The T₀ plants were kept for seed collection purpose, therefore GUS staining was performed only on leaf pieces. The plants were allowed to set seed. These seeds were used after harvest for the confirmation of expression pattern. (GUS expression in leaf tissue is listed in FIG. 2 to FIG. 8)

### Example 6: Histochemical GUS Assay

The plant material was incubated in GUS buffer for up to 16 hours at 37° C. (Jefferson et al. 1987). GUS Buffer Composition: [50 ml phosphate buffer, 10 ml 0.1% TritonX, 2 ml Potassium ferricyanate, 2 ml Potassium ferrocyanide, 20 ml methanol in 15 ml distilled water)+1 ml X-Gluc stock (50 mg X-Gluc in 1 ml DMF)]. Chlorophyll was extracted by washing with 70% ethanol (for 8 hours).

### Example 7: Stability of the Expression Patterns of the Promoters of the Present Invention in Multiple Generations

The above-mentioned analyses were performed on T₀ plants originating from the transformed tissues. The stability of promoter activity in the next generations or progeny plants of the original T₀ plant the so-called T₁ and T₂ plants was evaluated as follows. T₁ plants were analyzed as described above and the T₁ plants were allowed to reach maturity and to set T₂ seeds.

The expression pattern of the promoters of the present invention was studied in T₁ and T₂ plants as described. Number of events analyzed is mentioned in Table 4.

### Example 8: Expression Patterns of the Promoters of the Present Invention Under Different Stress Conditions

Arabidopsis events T₁ seeds were sown in sandy soil were kept in a culture room with light intensity maintained at 12,000 to 14,000 lux and with a 16-h-light/8-hr dark cycle at 25-28° C.

Stress Conditions:

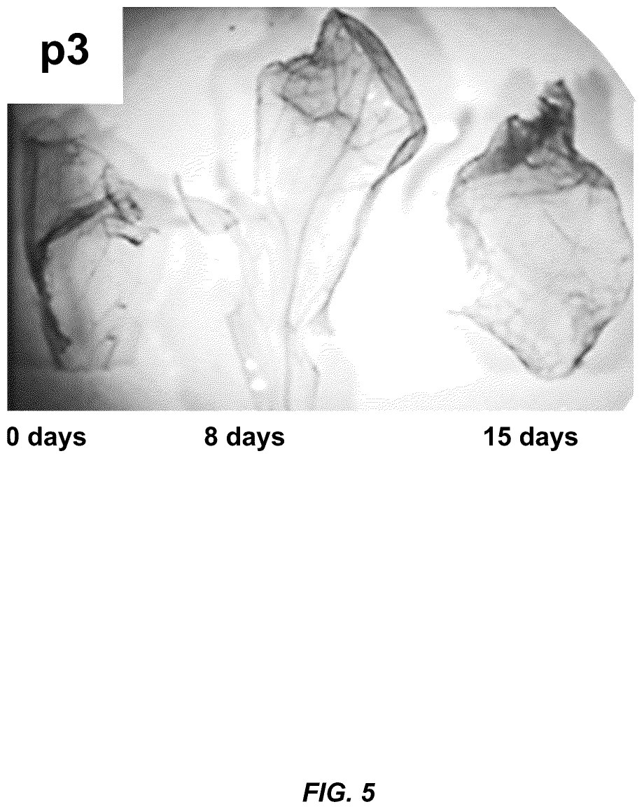

Sampling was done once before the start of the stress assay and after stress exposure of particular time. Stress assay details are mentioned in Table 5. For expression patterns of the promoters under various stress conditions refer FIGS. 5 to 12.

TABLE 4
Event analysis details

| | Promoter | |
|---|---|---|
| Sr. No. | ID | No. of T₀ event analyzed |
| 1 | P3 | 1 |
| | | (At./pMDCP3-2_Did quantification) |
| 2 | P32 | 3 |
| | | (At./pMDcP32-1, 2 and 7) |
| 3 | P44 | 7 |
| | | (At./pMDCP44-10, 18, 23, 25, 29, 30, 34) |

### Example 9: GUS Quantification

Quantification of GUS activity was performed by fluorometric assay described in Jefferson et al., 1987 (Jefferson et al., 1987, EMBO J., 6, 3901-3907) and Gallagher 1992 (Gallagher, S. R. (1992) Academic Press, Inc., New York, pp. 47-59).

Plant extract: 100 mg leaf tissues were ground in 200 μl of extraction buffer [50 mM NaPO₄ pH 7.0, 10 mM EDTA, 0.1% Triton X-100, 0.1% sodium lauryl sarcosine, and 10 mM β-mercaptoethanol]. The leaf tissue was then centrifuged at 12000 rpm for 15 minutes at 4° C. to remove cell debris. Supernatant was transferred to a fresh tube.

MUG assay: 20 μl homogenates (approximately μg of protein) were mixed with 80 μl of GUS assay buffer [2 mM MUG extraction buffer (to prepare 10 ml assay solution mix MUG 8.8 mg in extraction buffer). Prepare freshly just before use].

The mixture was vortexed and incubated at 37° C. for 30 minute. 2 μl of each reaction mixture and of each MU standard were mixed with 475 μl of stop buffer [200 mM Na₂CO₃ pH 11.2 (21.2 gm/L)]. 200 μl from above step were loaded by duplicated manner in a micro-titer plate and florescence were determined, excitation at 365 nm and emission at 444 nm.

Calculations of GUS Activity:

$$pico Mole MU / μ g of protein / minute = \frac{pico Mole MU / well}{0.5 μg of protein X minute of assay}$$

Concentrated MU calibration stock solution: Mix 9.9 mg in 50 ml D/W to prepare 1 mM MU stock. Make 1:10 dilution to get 10004 MU stock and 1:50 dilution to get 20 μM stock solution. For standard curve used 0, 4, 8, 12, 20, 40, 100, 250, 500 pmol MU.

### Example 10

Abiotic stress viz., salt stress, water stress, heat stress and cold stress assay conditions are mentioned in table 5 and GUS quantification data under different stress conditions, before stress and after stress, pmole MU/mg protein/min values along with fold change in expression is presented in tables for FIG. 9A-12B and Table 6.

TABLE 5
Stress assay conditions:

| Abiotic | |
|---|---|
| stress | |
| condition | Assay details |
| Salt stress | Around 2 months old plants of Arabidopsis events of respective promoter (at |
| | seed set stage) exposed to 150 mM NaCl stress for 2 hr and 5 hr. Samples were |
| | collected at 0 min, 2 hrs and 5 hrs of stress. |
| Water | Around 2 months old plants of Arabidopsis events of respective promoter (at |
| stress | seed set stage) exposed to water stress by withholding water for 8 and 15 days or |
| | 17 days. Samples were collected at 0 day, 8 days, and 15 or 17 days after |
| | water stress. |
| Heat | Around 2 months old plants of Arabidopsis events of respective promoter (at |
| Stress | seed set stage) exposed to Heat stress at 35° C. and 42° C. Samples were collected |
| | at 0 min, 2 hrs and 5 hrs of stress. |
| Cold | Around 2 months old plants of Arabidopsis events of respective promoter (at |
| stress | seed set stage) exposed to cold stress at 4° C. Samples were collected at 0 min, |
| | 2 hrs and 5 hrs of stress. |

Salt stress:P3 and P32 promoter

| Plant | | | | | | | | |
|---|---|---|---|---|---|---|---|---|
| code | 0 min | 2 hr | 5 hr | SE | 0 min | 2 hr | 5 hr | n |
| Control | <Min | 34 | <Min | Control | <Min | <Min | <Min | 3 |
| P3 | 3904 | 6309 | 20682 | P3 | 1915 | 677 | 101 | 2 |
| P32 | 10966 | 12618 | 18603 | P32 | 7410 | 7390 | 6347 | 3 |

Table for FIG. 9A

Salt stress:P44 promoter

| Plant | pMOL MU/mg protein/min | | | SE | | | |
|---|---|---|---|---|---|---|---|
| code | 0 min | 2 hr | 5 hr | 0 min | 2 hr | 5 hr | n |
| At./ | 28086 | 38292 | 49783 | 5368 | 9461 | 6608 | 3 |
| pMDCP44-23 | | | | | | | |
| At./ | 28711 | 33544 | 47351 | 2779 | 3413 | 14990 | 3 |
| pMDCP44-29 | | | | | | | |
| WT | <Min | <Min | <Min | <Min | <Min | <Min | 3 |

Table for FIG. 9B

Water stress:P3 and P32 promoter

| Plant | Water stress | | | SE | | | |
|---|---|---|---|---|---|---|---|
| code | 0 day | 8th day | 15th day | 0 day | 8th day | 15th day | n |
| Control | 0 | 0 | 0 | 0 | 0 | 0 | 3 |
| P3 | 2259 | 2455 | 1405 | 225 | 412 | 367 | 3 |
| P32 | 292 | 486 | 1134 | 175 | 176 | 40 | 2 |

Table for FIG. 10A

Table for FIG. 10B
Water stress: P44 promoter

| | pMOL MU/mg protein/ | | SE | | |
|---|---|---|---|---|---|
| | min | | | 17 | |
| Plant code | 0 Day | 17 Day | 0 Day | Day | n |
| At./pMDC P44-23 | 24966 | 42389 | 3385 | 5098 | 3 |
| WT | <Min | <Min | <Min | <Min | 3 |

Heat stress:At 35° C.:P3 and P32 promoter

| Plant | | | | | | | |
|---|---|---|---|---|---|---|---|
| code | 0 min | 2 hr | 5 hr | SE_0 min | 2 hr | 5 hr | n |
| WT | <Min | <Min | <Min | <Min | <Min | <Min | 3 |
| P3 | 1128 | 1315 | 1745 | 228 | 315 | 200 | 3 |
| P32 | 203 | 89 | 415 | 203 | 62 | 120 | 3 |

Table for FIG. 11A

Table for FIG. 11B
Heat stress: At 35° C.: P44 promoter

| | | pMOL MU/mg protein/min | SE | | |
|---|---|---|---|---|---|
| Plant code | 0 Min | 2 hr | 0 Min | 2 hr | n |
| At./pMDCP44- | 42613 | 23214 | 2602 | 3390 | 3 |
| 23 | | | | | |
| WT | <Min | <Min | <Min | <Min | 3 |

Cold stress:40 C.:P3 and P32 promoter

| Plant | | | | | | | |
|---|---|---|---|---|---|---|---|
| code | 0 min | 2 hr | 5 hr | SE | 0 min | 2 hr | 5 hr |
| WT | <Min | <Min | <Min | WT | <Min | <Min | <Min |
| p3 | 777 | 982 | 1556 | p3 | 106 | 75 | 78 |
| p32 | 154 | 263 | 1726 | p32 | 88 | 95 | 191 |

Table for FIG. 12A

Cold stress:At 4° C.:P44 promoter

| Plant | pMOL MU/mg protein/min | | | SE | | |
|---|---|---|---|---|---|---|
| code | 0 Min | 2 hr | 5 hr | 0 Min | 2 hr | 5 hr |
| At./pMDCP44-23 | 22291 | 24851 | 39926 | 1742 | 2278 | 7170 |
| WT | <Min | <Min | <Min | <Min | <Min | <Min |

Table for FIG. 12B

TABLE 6
GUS Quantification data before stress and after stress for P3, P32
and P44 promoter

| Pro- | Before Stress | After stress | | |
|---|---|---|---|---|
| moter | (pmole MU/mg protein/min) | | Fold ↑^{se} | Stress |
| P3 | 3903 | 20682 | 4.2 | Salt |
| P32 | 10965 | 18602 | 0.7 | stress |
| P44 | 28086 | 49783 | 0.8 | |
| P3 | 2259 | 1405 | App. 0.08-Fold ↑^{se} after | Water |
| | | | 8 days of stress and | Stress |
| | | | dropdown after 15 days | |
| | | | of stress | |
| P32 | 291 | 1133 | 2.9 | |
| P44 | 24966 | 42389 | 0.7 | |
| P3 | 777 | 1556 | 1.0 | Cold |
| P32 | 153 | 1725 | 10.27 | stress |
| P44 | 22291 | 39926 | 0.8 | |
| P3 | 1128 | 1745 | 0.54 | Heat |
| P32 | 203 | 415 | 1.04 | stress |
| P44 | 42613 | 23214 | Reduction in expression | |
| | | | after stress | |

### Example 11: Cis-Regulatory Element Analysis

All promoters were analyzed for the presence of cis-regulatory elements using PLACE database. (PLACE—Plant cis-acting regulatory DNA elements, http://www.dna.affrc.go.jp/PLACE/). Identified cis-regulatory elements of respective promoter are tabulated in table 7.

TABLE 7
Cis-regulatory element analysis of promoters

| Sr. | Promoter | |
|---|---|---|
| No. | ID | Cis-regulatory elements |
| 1 | P3 | -10PEHVPSBD, |
| | | ABRELATERD1, ABREOSRAB21, ACGTABOX, ACGTATERD1, |
| | | ANAERO1CONSENSUS, ARR1AT, ASFIMOTIFCAMV, |
| | | BIHD1OS, BOXIINTPATPB, BOXLCOREDCPAL, CAATBOX1, |
| | | CACTFTPPCA1, CAREOSREP1, CBFHV, CCA1ATLHCB1, CCAATBOX1, |
| | | CGACGOSAMY3, CIACADIANLELHC, CRTDREHVCBF2, |
| | | CURECORECR, DOFCOREZM, E2FCONSENSUS, EBOXBNNAPA, |
| | | ELRECOREPCRP1, GATABOX, GT1CONSENSUS, GT1CORE, |
| | | GT1GMSCAM4, GTGANTG10, HEXAT, IBOXCORE, |
| | | LTRECOREATCOR15, MARABOX1, MYB1AT, MYBATRD22, |
| | | MYBCORE, |
| | | MYBPZM, MYCCONSENSUSAT, NODCON2GM, OSE1ROOTNODULE, |
| | | POLASIG1, POLLENILELAT52, PRECONSCRHSP70A, |
| | | PYRIMIDINEBOXOSRAMY1A, RAV1AAT, RAV1BAT, |
| | | RBCSCONSENSUS, REALPHALGLHCB21, |
| | | RHERPATEXPA7, ROOTMOTIFTAPOX1, S1FBOXSORPS1L21, |
| | | SEF1MOTIF, SEF3MOTIFGM, SEF4MOTIFGM7S, SREATMSD, |
| | | SURECOREATSULTR11, TAAAGSTKST1, TATABOX2, TATABOX4, |
| | | TATABOX5, TATABOXOSPAL, TATCCAOSAMY, TBOXATGAPB, |
| | | TGACGTVMAMY, UPRMOTIFIIAT, |
| | | WBBOXPCWRKY1, WBOXATNPR1, WBOXHVISO1, WBOXNTERF3, |
| | | WRKY71OS |
| 2 | P32 | -10PEHVPSBD, |
| | | ABRELATERD1, ACGTABOX, ACGTATERD1, ANAERO3CONSENSUS, |
| | | ARFAT, ARR1AT, ASF1MOTIFCAMV, BIHD1OS, BOXIINTPATPB, |
| | | CAATBOX1, CACTFTPPCA1, |
| | | CARGCW8GAT, CATATGGMSAUR, CCAATBOX1, CEREGLUBOX2PSLEGA, |
| | | CPBCSPOR, CURECORECR, DOFCOREZM, DPBFCOREDCDC3, |
| | | EBOXBNNAPA, EECCRCAH1, GATABOX, GT1CONSENSUS, |
| | | GT1CORE, GT1GMSCAM4, |
| | | GTGANTG10, HDZIP2ATATHB2, IBOXCORE, INRNTPSADB, |
| | | LECPLEACS2, MARTBOX, MYB1AT, |
| | | MYBATRD22, MYBCORE, MYBST1, MYCATRD22, |
| | | MYCCONSENSUSAT, NODCON1GM, NODCON2GM, |
| | | NTBBF1ARROLB, |
| | | OSE1ROOTNODULE, OSE2ROOTNODULE, P1BS, POLASIG1, |
| | | POLASIG2, POLASIG3, POLLEN1LELAT52, |
| | | PYRIMIDINEBOXOSRAMY1A, QELEMENTZMZM13, RAV1AAT, |
| | | RBCSCONSENSUS, REALPHALGLHCB21, ROOTMOTIFTAPOX1, |
| | | SEF1MOTIF, SEF4MOTIFGM7S, |
| | | SORLIP1AT, SORLIP5AT, SP8BFIBSP8BIB, SREATMSD, SURECOREA |
| | | TSULTR11, SV40COREENHAN, |
| | | TAAAGSTKST1, TATABOX2, TATABOX3, TATABOX4, TATABOX5,. |
| | | TATABOXOSPAL, |
| | | TGACGTVMAMY, TGTCACACMCUCUMISIN, WBOXATNPR1, |
| | | WBOXHVISO1, WBOXNTERF3, WRKY71OS. |
| 3 | P44 | ABRELATERD1, ABRERATCAL, ACGTATERD1, ACGTTBOX, |
| | | MYB1AT, MYB2AT, MYB2CONSENSUSAT, MYBCORE, |
| | | MYCATERD1, MYCCONSENSUSAT |
| | | GT1GMSCAM4 |
| | | BIHD1OS, WBOXATNPR1: “W-box” found in promoter of Arabidopsis |
| | | thaliana (A.t.) NPR1gene; Located between +70 and +79 in tandem; They |
| | | were recognized specifically by salicylic acid (SA)-induced WRKY DNA |
| | | binding proteins, WRKY71OS, ELRECOREPCRP1ElRE (Elicitor |
| | | Responsive Element) core of parsley (P.c.) PR1genes; consensus sequence |
| | | of elements W1 and W2 of parsley PR1-1and PR1-2 promoters; Box W1 |
| | | and W2 are the binding site of WRKY1and WRKY2, respectively; |
| | | LTRE1HVBLT49 |
| | | AACACOREOSGLUB1, CANBNNAPA, EBOXBNNAPA, |
| | | GCN4OSGLUB1, SEF4MOTIFGM7S, DOFCOREZM, |
| | | DPBFCOREDCDC3 A novel class of bZIP transcription factors, DPBF-1 |
| | | and 2 (Dc3promoter-binding factor-1 and 2) binding core sequence; Found |
| | | in the carrot (D.c.) Dc3 gene promoter; Dc3 expression is normally |
| | | embryo-specific, and also can be induced by ABA |
| | | CATATGGMSAUR auxin responsiveness, ARFAT response to auxin, |
| | | CAREOSREP1, GAREAT Two cis-acting elements necessary and |
| | | sufficient for gibberellin-upregulated proteinase expression in rice seeds |
| | | GATA, |
| | | MYBST1, |
| | | AGMOTIFNTMYB2: AG-motif found at −114 of the promoter of NtMyb2 |
| | | gene; NtMyb2 is a regulator of the tobacco retrotransposon Tto1 and the |
| | | defence-related gene phenylalanine ammonia lyase (PAL), which are |
| | | induced by various stress such as wounding or elicitor treatment; AGP1 |
| | | (GATA-type zinc finger protein) binding site. |
| | | HBOXCONSENSUSPVCHS “H-box”; Consensus sequence of H-boxes |
| | | found in bean (Phaseolus vulgaris) chs15 gene promoter; Essential for both |
| | | light regulation and elicitor induction; Similar sequence was found in |
| | | tobacco Tntl retrotransposon promoter (LTR); Tnt1 is induced by |
| | | wounding and by abiotic stress; “KAP-2” binds to the H-box and |
| | | stimulates transcription from a promoter harboring the H-box; |

What is claimed:

1) A stress inducible promoter comprising a nucleic acid sequence as set forth in SEQ ID NO. 1 or complements thereof or SEQ ID No.2 or complements thereof.

2) The promoter as claimed in claim 1, wherein the promoter is an abiotic stress inducible promoter.

3) The promoter as claimed in claim 2, wherein the promoter is induced by salt, water, cold or heat stress.

4) A stress inducible promoter comprising a nucleic acid sequence as set forth in SEQ ID NO. 3 or complements thereof.

5) The promoter as claimed in claim 4, wherein the promoter is an abiotic stress inducible promoter.

6) The promoter as claimed in claim 5, wherein the promoter is induced by salt, water, or cold stress.

7) The promoter as claimed in claim 1, wherein the promoter is derived from Gossypium hirsutum.

8) The promoter as claimed in claim 4, wherein the promoter is derived from Gossypium hirsutum.

9) A genetic construct comprising the promoter as claimed in claim 1 operably linked to a heterologous nucleic acid sequence of interest.

10) A genetic construct comprising the promoter as claimed in claim 4 operably linked to a heterologous nucleic acid sequence of interest.

11) A recombinant plant expression vector comprising the genetic construct of claim 9.

12) A recombinant plant expression vector comprising the genetic construct of claim 10.

13) A host cell comprising the promoter comprising a nucleic acid sequence as set forth in SEQ ID No.1 or SEQ ID No.2 or SEQ ID No.3 or complements thereof or genetic construct comprising the promoter comprising a nucleic acid sequence as set forth in SEQ ID No.1 or SEQ ID No.2 or SEQ ID No.3 or complements thereof operably linked to a heterologous nucleic acid sequence of interest or vector comprising the genetic construct.

14) A transgenic plant comprising host cell as claimed in claim 13, wherein the transgenic plant is a monocot or a dicot plant.

15) A transgenic seed produced from transgenic plant of claim 14, wherein the transgenic seed is a monocot or a dicot seed.

16) A method of expressing a heterologous nucleic acid of interest in a plant comprising introducing the genetic construct comprising the promoter comprising a nucleic acid sequence as set forth in SEQ ID NO.1 or SEQ ID No.2 or SEQ ID No.3 or complements thereof operably linked to a heterologous nucleic acid sequence of interest, wherein the promoter is induced by an abiotic stress.

17) The method as claimed in claim 16, wherein the promoter having the nucleic acid sequence as set forth in SEQ ID NO:1, SEQ ID No. 2 or complements thereof is induced by salt, water, cold or heat stress.

18) The method as claimed in claim 16, wherein the promoter having the nucleic acid sequence as set forth in SEQ ID NO.3 or complements thereof is induced by salt, water, or cold stress.

19) The method as claimed in claim 16, wherein the heterologous nucleic acid of interest is expressed in the whole plant.
